(19) United States
(12) Patent Application Publication
(10) Pub. No.: US 2024/0215883 A1
(43) Pub. Date: Jul. 4, 2024

(54) INFORMATION PROCESSING APPARATUS, INFORMATION PROCESSING METHOD, AND PROGRAM

(71) Applicant: SONY GROUP CORPORATION, TOKYO (JP)

(72) Inventor: KIMINOBU NISHIMURA, TOKYO (JP)

(21) Appl. No.: 18/555,874

(22) PCT Filed: Jan. 13, 2022
(86) PCT No.: PCT/JP2022/000905

§ 371 Date: Oct. 18, 2023

(30) Foreign Application Priority Data
May 7, 2021 (JP) 2021-078943

(51) Int. Cl. A61B 5/16; G16H 50/20

(52) U.S. Cl. CPC A61B 5/165; G16H 50/20

(57) ABSTRACT

To sense a sign of a predetermined state at an early stage. Provided is an information processing apparatus including a state sensing unit that senses a change in state of a reactant on the basis of a time-series record of reactions made by the reactant to at least one predetermined input pattern executed by an input body, in which the predetermined input pattern is an event that repeatedly occurs in an environment in which the reactant lives.

### TECHNICAL FIELD

The present disclosure relates to an information processing apparatus, an information processing method, and a program.

### BACKGROUND ART

In recent years, there has been developed technology for sensing a state of an object on the basis of acquired sensor information. For example, Patent Document 1 discloses a system that compares biological data acquired from a sleeping subject with biological data acquired from a sleeping dementia patient to determine a risk of dementia for the test subject.

### CITATION LIST

### Patent Document

Patent Document 1: Japanese Patent Application Laid-Open No. 2016-22310

### SUMMARY OF THE INVENTION

### Problems to be Solved by the Invention

However, in a determination method as disclosed in Patent Document 1, unless a symptom of dementia with the test subject progresses at an equivalent level to a symptom of dementia with the dementia patient does, it is expected that the biological data does not show a strong characteristic of dementia. Therefore, with the determination method as disclosed in Patent Document 1, it is difficult to sense a sign of dementia at an early stage.

### Solutions to Problems

According to one aspect of the present disclosure, there is provided an information processing apparatus including a state sensing unit that senses a change in state of a reactant on the basis of a time-series record of reactions made by the reactant to at least one predetermined input pattern executed by an input body, in which the predetermined input pattern is an event that repeatedly occurs in an environment in which the reactant lives.

Furthermore, according to another aspect of the present disclosure, there is provided an Information processing method including, by a processor, sensing a change in state of a reactant on the basis of a time-series record of reactions made by the reactant to at least one predetermined input pattern executed by an input body, in which the predetermined input pattern is an event that repeatedly occurs in an environment in which the reactant lives.

Furthermore, according to another aspect of the present disclosure, there is provided a program for causing a computer to function as an information processing apparatus including a state sensing unit that senses a change in state of a reactant on the basis of a time-series record of reactions made by the reactant to at least one predetermined input pattern executed by an input body, in which the predetermined input pattern is an event that repeatedly occurs in an environment in which the reactant lives.

### BRIEF DESCRIPTION OF DRAWINGS

FIG. 1 is a diagram for describing an overview of sensing a sign of a mental illness according to an embodiment of the present disclosure.

FIG. 2 is a block diagram illustrating an example of a system configuration related to recording of an input pattern and reaction according to the embodiment.

FIG. 3 is a diagram illustrating an example of information stored in an input-reaction DB 220 according to the embodiment.

FIG. 4 is a flowchart illustrating an example of a flow of operation of recording of an input pattern and reaction according to the embodiment.

FIG. 5 is a block diagram illustrating an example of a system configuration related to presentation control based on sensing of a change in state of a reactant and a result of the sensing according to the embodiment.

FIG. 6 is a diagram illustrating an example of an interface for performing various settings for sensing of a change in state of a reactant according to the embodiment.

FIG. 7 is a diagram illustrating an example of sensing a change in state of the reactant and the presentation control according to the embodiment.

FIG. 8 is a diagram illustrating an example of sensing a change in state of the reactant and the presentation control according to the embodiment.

FIG. 9 is a diagram illustrating an example of knowledge of a sign of a mental illness according to the embodiment.

FIG. 10 is a diagram for describing a configuration of recording of information including diagnostic information according to the embodiment.

FIG. 11 is a flowchart illustrating an example of a flow of learning by a state sensing unit 230 according to the embodiment.

FIG. 12 is a diagram illustrating an example of data used for clustering according to the embodiment.

FIG. 13 is a diagram illustrating a result of clustering of data illustrated in FIG. 12 and an example of presentation based on the result according to the embodiment.

FIG. 14 is a diagram illustrating an example of a checklist according to an embodiment of the present disclosure.

FIG. 15 is an example of an interface for schedule reservation according to the embodiment.

FIG. 16 is an example of an interface for access management of the reactant according to the embodiment.

FIG. 17 is a diagram illustrating an example of an interface in a case where a system according to the embodiment is used in a general home.

FIG. 18 is a diagram illustrating an example of an interface in a case where the system according to the embodiment is used in the general home.

FIG. 19 is a diagram for describing an example in a case where the system according to the embodiment is applied to an online class or the like.

FIG. 20 is a diagram illustrating an example of an interface that presents information regarding sensing of abuse according to the embodiment.

FIG. 21 is a diagram illustrating an example of an interface that presents information regarding sensing of abuse according to the embodiment.

FIG. 22 is a block diagram illustrating a hardware configuration example of an information processing apparatus 90 according to an embodiment of disclosure according to the embodiment.

### MODE FOR CARRYING OUT THE INVENTION

A preferred embodiment of the present disclosure will now be described in detail with reference to the accompanying drawings. Note that in the present specification and drawings, components having substantially the same functional configuration are denoted using the same reference numerals. Redundant explanations are therefore omitted.

Note that the description will be made in the following order.
1. Embodiment
1.1. Overview
1.2. Recording of input pattern and reaction
1.3. State change sensing and presentation control
1.4. Application example
2. Hardware configuration example
3. Conclusion

### <1. EMBODIMENT>

### 1.1. Overview

As described above, there has been developed technology for sensing a state of an object on the basis of acquired sensor information.

Examples of the object described above include animals including humans.

Furthermore, the state of the object includes a mental state of the object.

Moreover, the mental state described above may include various mental illnesses.

Examples of the mental illness include dementia, attention-deficit hyperactivity disorder (ADHD), schizophrenia, depression, and the like.

In the above-described mental illnesses, the more the symptoms progress, the more difficult the treatment is, and therefore, it is important to sense a sign of onset at an early stage and to perform appropriate care.

For this purpose, in recent years, techniques for sensing a risk of a mental illness as described above have also been developed.

However, as in a technique disclosed in Patent Document 1, for example, many of the techniques as described above merely sense a specific state (including words and actions) that may appear in a certain mental illness.

However, it is expected that the specific state as described above does not appear in a case where the mental illness has not yet progressed to some extent.

For this reason, for example, it is difficult to sense a sign of a mental illness at an early stage only by comparing a state of a test subject with a state specific to a certain mental illness as described above.

A technical idea according to an embodiment of the present disclosure has been conceived by focusing on the above-described points, and enables sensing of a sign of a predetermined state of an object at an early stage.

In order to achieve the above, an embodiment of the present disclosure focuses on a time-series change in reactions made by a test subject (hereinafter, referred to as a reactant) to a certain input.

For example, for a mental illness such as dementia, it has been reported that a change in mental state, such as becoming irritable or feeling anxious, may occur as an initial symptom.

Therefore, an information processing apparatus 20 that implements an information processing method according to the present embodiment may sense a change in state of the reactant on the basis of a record of the time-series change in reactions made by the reactant.

However, it is expected that the reactions made by the reactant have different characteristics depending on factors that cause the reactions.

As an example, regardless of having a mental illness, there is a high possibility that a reaction of the reactant in a case where another party uses a polite language is different in terms of characteristics, from a reaction of the reactant in a case where another party uses a disrespectful language.

Therefore, in a case where reactions to different factors are not differentiated, accuracy of sensing the state change may be significantly degraded.

In order to avoid a degradation in accuracy of sensing the above-described state change, the information processing apparatus 20 according to the present embodiment may include a state sensing unit 230 that senses a change in state of a reactant on the basis of a time-series record of reactions made by the reactant to at least one predetermined input pattern executed by an input body.

Furthermore, one of the characteristics of the above-described predetermined input pattern is an event that repeatedly occurs in an environment in which the reactant lives.

According to the above-described configuration, it is possible to sense a change in state of the reactant with high accuracy by tracking reactions made by the reactant for each predetermined input pattern repeatedly occurs in daily life.

Here, an example of the input pattern according to the present embodiment includes words and actions performed by the input body to the reactant.

More specifically, the input pattern according to the present embodiment may be, for example, a greeting, request, question, or the like by the input body to the reactant.

By tracking the reactions made by the reactant for each input pattern expected to occur frequently in daily life as described above, the information processing apparatus 20 according to the present embodiment can sense a change in state of the reactant without increasing burden on the input body and reactant.

Hereinafter, there will be described an overview of the information processing apparatus 20 sensing a change in state of a reactant according to the present embodiment.

Note that, hereinafter, a case where the reactant according to the present embodiment is a care receiver will be described as an example.

As an example, the reactant according to the present embodiment may be a resident of a nursing home.

In this case, the input body according to the present embodiment may be a nursing care staff who cares for the above-described resident.

Furthermore, in the following, there will be exemplified a case where the state sensing unit 230 included in the information processing apparatus 20 according to the present embodiment senses a change in mental state of the reactant on the basis of a time-series record of reactions made by the reactant.

More specifically, the state sensing unit 230 according to the present embodiment may sense a sign of a mental illness of the reactant on the basis of the time-series record of the reactions made by the reactant.

Here, the above-described mental illnesses may include, for example, dementia, attention-deficit hyperactivity disorder, schizophrenia, depression, and the like.

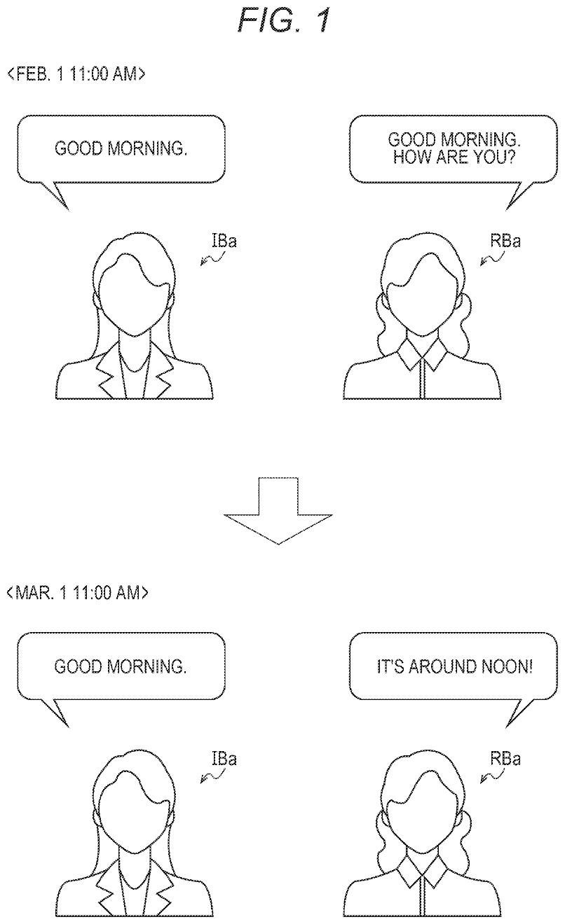

FIG. 1 is a diagram for describing an overview of sensing a sign of a mental illness according to an embodiment of the present disclosure.

The upper part of FIG. 1 illustrates an example of a reaction made by a reactant RBa, who is a resident of a nursing home, to a predetermined pattern performed by an input body IBa, who is a nursing care staff, at 11:00 AM on February 1.

Here, the above-described predetermined pattern may be a morning greeting.

In a case of an example illustrated in the upper part of FIG. 1, the reactant RBa gently reacts to the morning greeting by the input body IBa.

Meanwhile, the lower part of FIG. 1 illustrates an example of a reaction made by the reactant RBa to the predetermined pattern performed by the input body IBa at 11:00 AM on March 1, which is one month later.

In a case of an example illustrated in the lower part of FIG. 1, the reactant RBa reacts with irritation to the morning greeting by the same input body IBa.

In a system according to the present embodiment, as illustrated in FIG. 1, reactions made by a certain reactant to the same input pattern are recorded in time series.

Furthermore, the information processing apparatus 20 according to the present embodiment refers to the reactions of the reactant to a certain input pattern recorded in time series, and senses a change in mental state of the reactant, particularly a sign of a mental illness.

For example, in a case of an example illustrated in FIG. 1, the state sensing unit 230 included in the information processing apparatus 20 senses that the reactant RBa who had gently reacted to the morning greetings reacted with irritation on March 1.

Furthermore, the state sensing unit 230 can sense from the above-described reaction with irritation that the reactant RBa is irritable and further sense that, on the basis of the reaction being one of an initial symptom of dementia, there is a possibility of dementia with the reactant RBa.

Hereinafter, a configuration for achieving the above-described sensing will be described in detail.

### 1.2. Recording of Input Pattern and Reaction

In order to achieve sensing as described with reference to FIG. 1, there are required an input pattern performed by the input body and a mechanism for recording, in time series, reactions made by the reactant to the input pattern.

Therefore, first, among configurations included in the system according to the present embodiment, configurations related to the above-described recording will be described.

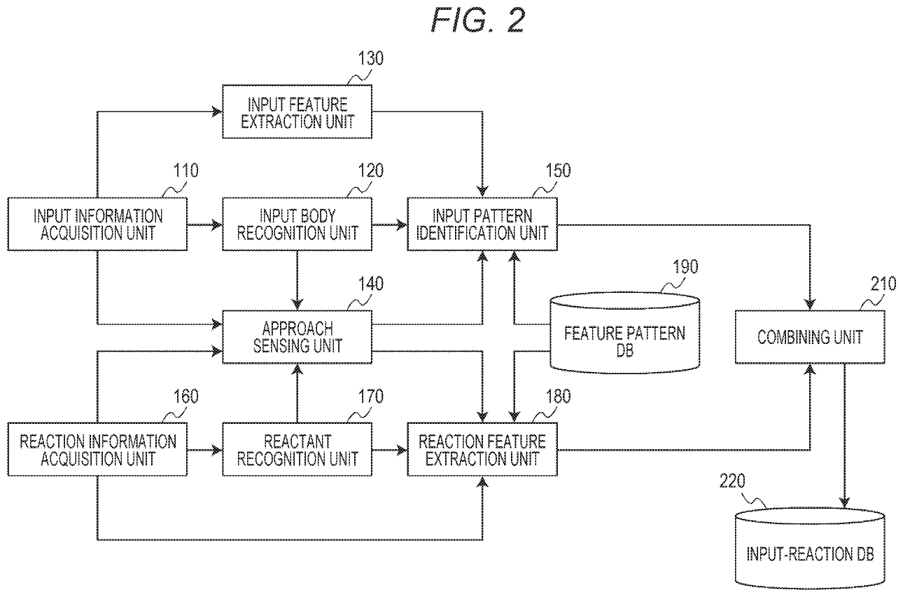

FIG. 2 is a block diagram illustrating an example of a system configuration related to recording of an input pattern and reaction according to the present embodiment.

As illustrated in FIG. 2, the system according to the present embodiment may include an input information acquisition unit 110, an input body recognition unit 120, an input feature extraction unit 130, an approach sensing unit 140, and an input pattern identification unit 150.

Furthermore, the system according to the present embodiment may include a reaction information acquisition unit 160, a reactant recognition unit 170, a reaction feature extraction unit 180, a feature pattern DB 190, a combining unit 210, and an input-reaction DB.

### (Input Information Acquisition Unit 110)

The input information acquisition unit 110 according to the present embodiment acquires information regarding an input pattern performed by the input body.

For this purpose, the input information acquisition unit 110 according to the present embodiment includes various sensors.

Examples of the above-described sensor include an image sensor, a microphone, an infrared sensor, a beacon, a biological sensor, and the like.

The input information acquisition unit 110 may be implemented as, for example, a wearable device worn by the input body.

Meanwhile, the input information acquisition unit 110 may be implemented as a monitoring camera or the like equipped in a room.

A form of the input information acquisition unit 110 according to the present embodiment can be flexibly modified according to the input body, the reactant, a target to be sensed by the state sensing unit 230, a characteristic of an environment to which the system is applied, and the like.

### (Reaction Information Acquisition Unit 160)

The reaction information acquisition unit 160 according to the present embodiment acquires information regarding a reaction made by the reactant.

The input information acquisition unit 110 and reaction information acquisition unit 160 according to the present embodiment may have equivalent configurations and functions, although targets from which information is acquired are different.

Therefore, detailed description of the reaction information acquisition unit 160 will be omitted.

### (Input Body Recognition Unit 120)

The input body recognition unit 120 according to the present embodiment identifies the input body on the basis of the information acquired by the input information acquisition unit 110.

For example, the input body recognition unit 120 may recognize the input body by comparing an image acquired by the input information acquisition unit 110 with an image of a pre-stored face of the input body.

Furthermore, for example, the input body recognition unit 120 may recognize the input body by comparing a characteristic of sound acquired by the input information acquisition unit 110 with a pre-stored characteristic of voice of the input body.

The input body recognition unit 120 may recognize the input body by using widely used recognition technology.

### (Reactant Recognition Unit 170)

The reactant recognition unit 170 according to the present embodiment identifies the reactant on the basis of the information acquired by the reaction information acquisition unit 160.

The input body recognition unit 120 and reactant recognition unit 170 according to the present embodiment may have equivalent configurations and functions, although objects to be identified are different.

Therefore, detailed description of the reactant recognition unit 170 will be omitted.

### (Input Feature Extraction Unit 130)

The input feature extraction unit 130 according to the present embodiment extracts a feature value from the information acquired by the input information acquisition unit 110.

For example, in a case where the information acquired by the input information acquisition unit 110 is sound, the input feature extraction unit 130 may execute sound recognition on the sound and extract text of “Good morning” or the like as the feature value.

Furthermore, for example, the input feature extraction unit 130 may perform frequency analysis on the sound described above and extract a cepstrum waveform value or the like as a feature value.

Furthermore, for example, in a case where the information acquired by the input information acquisition unit 110 is an image, the input feature extraction unit 130 may perform face detection on the image and extract a feature value regarding expression, color tone, emotion, or the like.

The input feature extraction unit 130 according to the present embodiment may extract various feature values by using widely used feature extraction technology.

### (Approach Sensing Unit 140)

The approach sensing unit 140 according to the present embodiment senses an approach between the input body and the reactant on the basis of the information acquired by the input information acquisition unit 110, a result of the recognition by the input body recognition unit 120, the information acquired by the reaction information acquisition unit 160, a result of recognition by the reactant recognition unit 170, or the like.

For example, the approach sensing unit 140 may sense the approach between the input body and the reactant on the basis of the recognized input body and reactant being located within a predetermined distance.

Furthermore, for example, the approach sensing unit 140 may sense the approach between the input body and the reactant on the basis of the recognized input body calling a name of the reactant.

On the basis of the wearable device worn by the input body sensing a beacon provided in a room assigned to a certain reactant, entry of the input body into the room of the reactant, that is, an approach between the input body and the reactant, may be sensed.

### (Input Pattern Identification Unit 150)

The input pattern identification unit 150 according to the present embodiment identifies the input pattern on the basis of the feature value extracted by the input feature extraction unit 130 and a feature value of a predetermined input pattern stored in the feature pattern DB 190.

For example, in a case where the feature pattern DB 190 stores text similar to the text “Good morning” extracted by the input feature extraction unit 130 in association with a “morning greeting”, the input pattern identification unit 150 can identify that the input pattern is a “morning greeting”.

Furthermore, for example, in a case where text similar to text “Let me take your temperature, please” extracted by the input feature extraction unit 130 is stored in the feature pattern DB 190 in association with a “request”, the input pattern identification unit 150 can identify that the input pattern is a “request”.

Furthermore, for example, in a case where text similar to text “Did you sleep well?” extracted by the input feature extraction unit 130 is stored in the feature pattern DB 190 in association with a “question”, the input pattern identification unit 150 can identify that the input pattern is a “question”.

Note that the input pattern identification unit 150 according to the present embodiment may identify an input pattern as described above in a case where an approach between the input body and the reactant is sensed by the approach sensing unit 140.

### (Reaction Feature Extraction Unit 180)

The reaction feature extraction unit 180 according to the present embodiment extracts a feature value of the reaction made by the reactant, on the basis of the information acquired by the reaction information acquisition unit 160, the result of the recognition by the reactant recognition unit 170, or the like.

Furthermore, at this time, the reaction feature extraction unit 180 according to the present embodiment may refer to feature values of various reactions, the feature values being stored in the feature pattern DB 190.

The input reaction feature extraction unit 180 according to the present embodiment may extract various feature values by using widely used feature extraction technology.

As an example, it is assumed a case where the reaction feature extraction unit 180 extracts a reaction speed with respect to the input pattern “morning greeting” as a feature value.

In this case, first, the input feature extraction unit 130 extracts, as a feature value, text of “Good morning” or the like from an utterance of the input body recognized by the input body recognition unit 120, and stores an end time Ta of the utterance.

Next, the reaction feature extraction unit 180 stores a start time Tb of a reaction utterance of the reactant recognized by the reactant recognition unit 170.

Next, the reaction feature extraction unit 180 extracts a difference between the above-described start time Tb and the end time Ta as a feature value of the reaction speed.

Note that the reaction feature extraction unit 180 according to the present embodiment may perform feature extraction as described above in a case where the approach sensing unit 140 senses an approach between the input body and the reactant.

### (Feature Pattern DB 190)

The feature pattern DB 190 according to the present embodiment is a database that stores a predetermined input pattern and feature values of various reactions.

### (Combining Unit 210)

The combining unit 210 according to the present embodiment combines the input pattern identified by the input pattern identification unit 150, the feature value of the reaction extracted by the reaction feature extraction unit 180, and the like, and stores them in the input-reaction DB 220.

### (Input-Reaction DB 220)

The input-reaction DB 220 according to the present embodiment is a database that stores information combined by the combining unit 210.

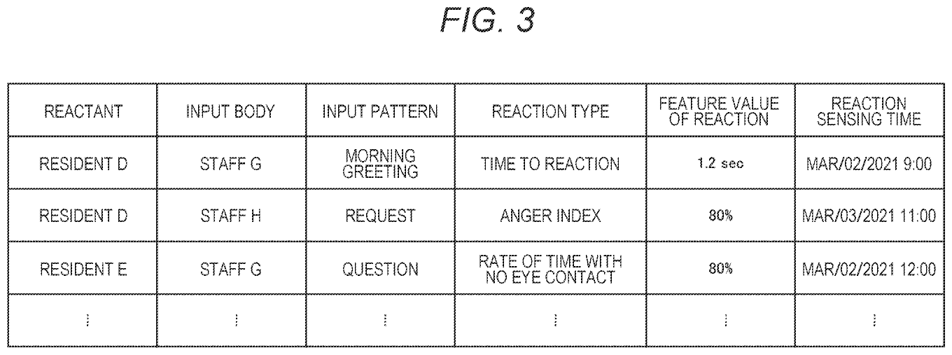

FIG. 3 is a diagram illustrating an example of information stored in an input-reaction DB 220 according to the present embodiment.

In a case of an example illustrated in FIG. 3, the recognized reactant, the recognized input body, the identified input pattern, a reaction type, the feature value of the reaction, reaction sensing time, and the like are stored in the input-reaction DB 220 in a combined manner.

A configuration example related to recording of an input pattern and reaction according to the present embodiment has been described above.

Each configuration described above may be implemented in a plurality of apparatuses in a distributed manner. Each configuration may transmit and receive information via wireless or wired communication.

Furthermore, the configuration described above described with reference to FIG. 2 is merely an example, and the configuration of the system according to the present embodiment is not limited to such an example.

The configuration of the system according to the present embodiment can be flexibly modified according to specifications and operations.

Subsequently, there will be described a flow of operation of recording of an input pattern and reaction according to the present embodiment.

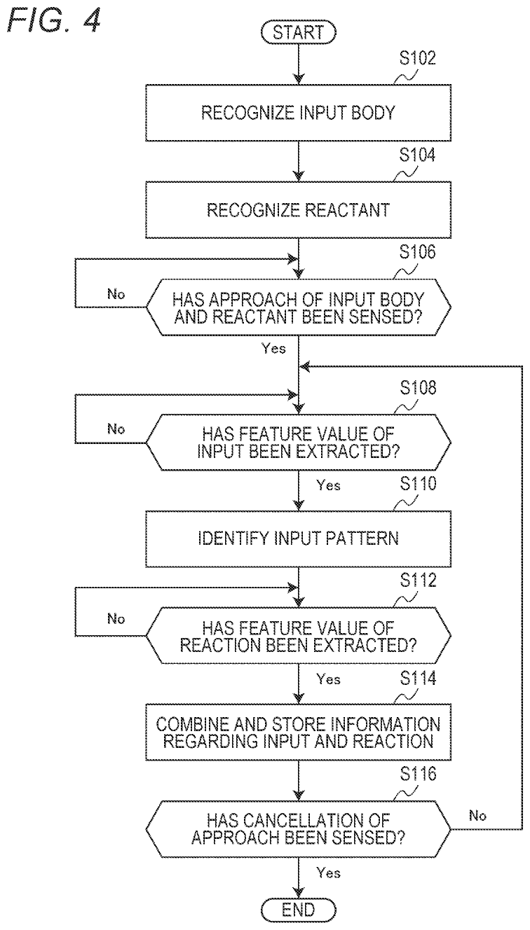

FIG. 4 is a flowchart illustrating an example of a flow of operation of recording of an input pattern and reaction according to the present embodiment.

In a case of an example illustrated in FIG. 1, first, the input body recognition unit 120 recognizes the input body (S102).

The input body recognition unit 120 may recognize the input body by the above-described various recognition methods.

Next, the reactant recognition unit 170 recognizes the reactant (S104).

The reactant recognition unit 170 may recognize the reactant with the above-described various recognition methods.

Subsequently, the approach sensing unit 140 determines whether or not the input body and the reactant have approached each other (S106).

The approach sensing unit 140 may repeatedly execute processing in Step S106 until an approach of the input body and the reactant is sensed.

Note that the approach sensing unit 140 may sense an approach between the input body and the reactant with the above-described various methods.

In a case where the approach sensing unit 140 senses an approach between the input body and the reactant (S106: Yes), the input feature extraction unit 130 subsequently attempts to extract a feature value of the input (S108).

The input feature extraction unit 130 may extract various feature values by using widely used technology.

Here, in a case where the input feature extraction unit 130 extracts the feature value of the input (S108: Yes), the input pattern identification unit 150 identifies the input pattern on the basis of the extracted feature value (S110).

Next, the reaction feature extraction unit 180 attempts to extract the feature value of the reaction (S112).

The reaction feature extraction unit 180 may extract various feature values by using widely used technology.

Here, in a case where the feature value of the reaction is extracted by the reaction feature extraction unit 180 (S112: Yes), the combining unit 210 combines information regarding the input and information regarding the reaction and stores the combined information in the input-reaction DB 220 (S114).

The processing in Steps S108 to S114 may be repeatedly executed until the approach sensing unit 140 senses cancellation of the approach between the input body and the reactant (S116: Yes).

### 1.3. State Change Sensing and Presentation Control

Next, there will be described sensing of state conversion of the reactant according to the present embodiment and presentation control based on a result of the sensing.

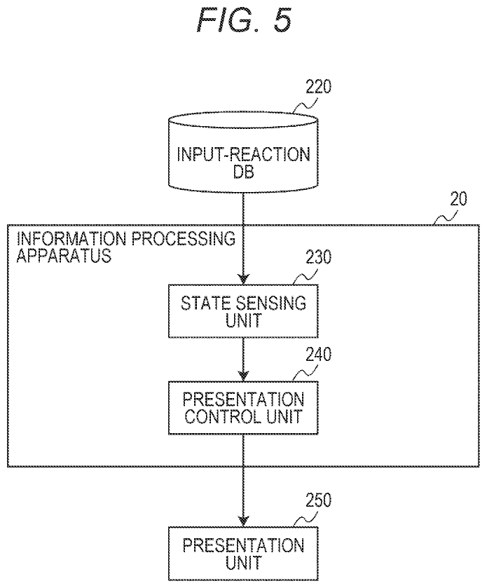

FIG. 5 is a block diagram illustrating an example of a system configuration related to presentation control based on sensing of a change in state of the reactant and a result of the sensing according to the present embodiment.

As illustrated in FIG. 5, the system according to the present embodiment includes the state sensing unit 230, a presentation control unit 240, and a presentation unit 250 in addition to each configuration described with reference to FIG. 2.

The state sensing unit 230 and presentation control unit 240 according to the present embodiment may be included in the information processing apparatus 20.

### (State Sensing Unit 230)

The state sensing unit 230 according to the present embodiment senses a change in state of the reactant on the basis of a time-series record of the reactions made by the reactant to at least one predetermined input pattern executed by an input body.

The state sensing unit 230 may acquire information regarding the above-described record from the input-reaction DB 220 and execute sensing of a state change on the basis of the information.

Details of functions of the state sensing unit 230 according to the present embodiment will be described later.

### (Presentation Control Unit 240)

The presentation control unit 240 according to the present embodiment controls presentation of a result of sensing by the state sensing unit 230.

A specific example of the presentation control by the presentation control unit 240 according to the present embodiment will be described later.

### (Presentation Unit 250)

The presentation unit 250 according to the present embodiment presents various types of information under control of the presentation control unit 240.

For this purpose, the presentation unit 250 according to the present embodiment includes various displays and speakers.

Next, sensing of the state change of the reactant according to the present embodiment and presentation control based on a result of the sensing will be described in detail with reference to specific examples.

First, various settings for sensing a change in state of the reactant according to the present embodiment will be described.

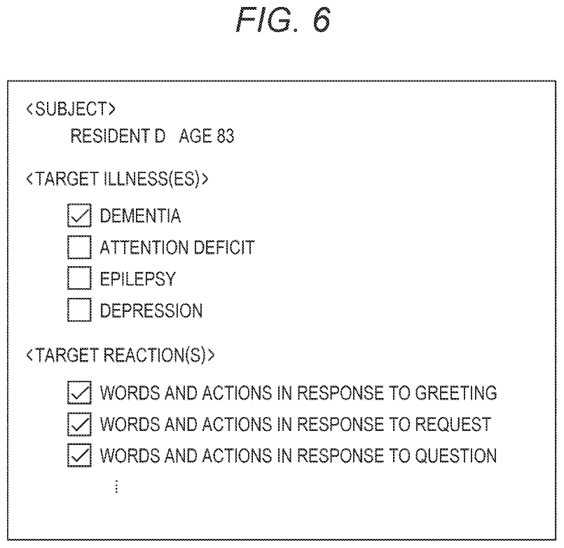

FIG. 6 is a diagram illustrating an example of an interface for performing various settings for sensing of a change in state of a reactant according to the present embodiment.

The presentation control unit 240 may control operation of an interface illustrated in FIG. 6.

For example, a user (here, an administrator who performs various settings for sensing a change in state of a reactant) may be able to set a reactant to be sensed, a mental illness to be sensed, a reaction to be sensed, and the like, by using the interface as illustrated in FIG. 6.

For example, in a case of an example illustrated in FIG. 6, the user performs settings for sensing a sign of “dementia” from a “resident D” on the basis of “words and actions in response to greeting”, “words and actions in response to request”, and “words and actions in response to question”.

The user can set each item as described above by using a check box or the like disposed in the interface.

Each configuration including the state sensing unit 230 according to the present embodiment may operate on the basis of the settings input as described above.

Next, there will be illustrated examples of sensing a change in state of the reactant according to the present embodiment and the presentation control according to the embodiment.

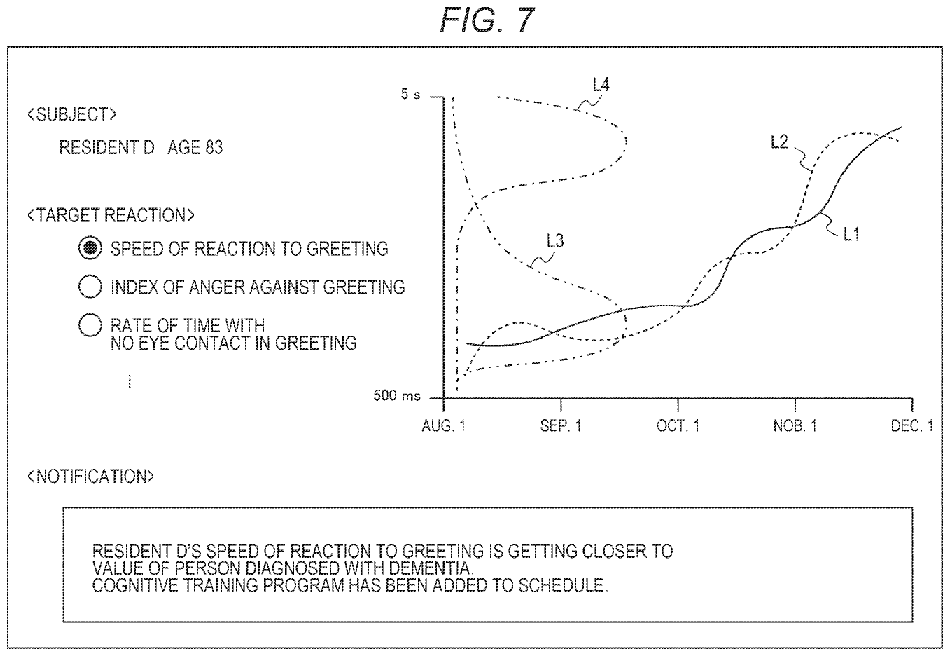

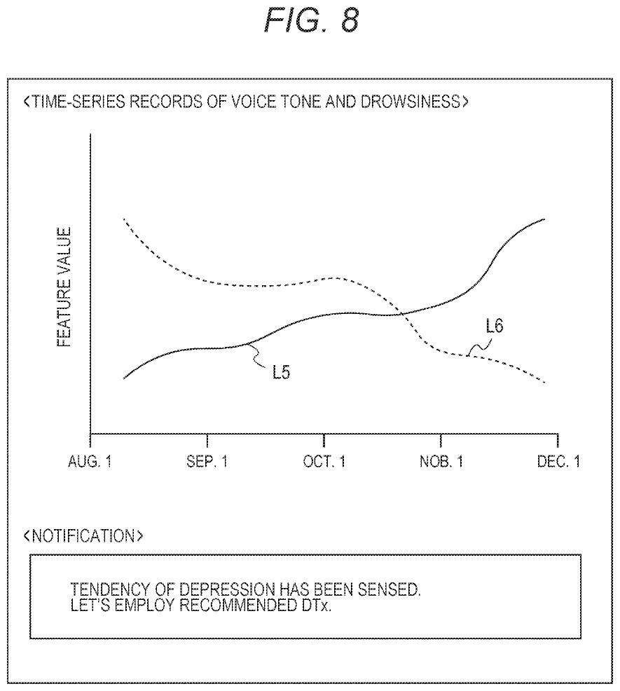

FIGS. 7 and 8 are diagrams illustrating examples of sensing a change in state of the reactant according to the present embodiment and the presentation control according to the embodiment.

The presentation control unit 240 may cause the presentation unit 250 to display an interface as exemplified in FIG. 7 or FIG. 8.

In a case of an example illustrated in FIG. 7, the interface displays a graph indicating a time-series record of the “reaction speed” of the “resident D” to an input pattern “greeting”, and a notification regarding a sign of dementia sensed by the state sensing unit 230 on the basis of the record.

Note that the user may switch the displayed graph by selecting another target reaction by using the interface.

As described above, the presentation control unit 240 according to the present embodiment may control presentation of a time-series record of reactions made by the reactant to the predetermined input pattern.

A curve L1 in the graph exemplified in FIG. 7 indicates a time-series record of reaction speed of the “resident D” with respect to the input pattern “greeting” by a certain input body (for example, a nursing care staff G).

Furthermore, a curve L2 in the graph exemplified in FIG. 7 indicates a time-series record of reaction speed of the “resident D” with respect to the input pattern “greeting” by another certain input body (for example, a nursing care staff H) different from the input body related to the curve L1.

As described above, the time-series record according to the present embodiment may be presented for each input body.

Furthermore, the state sensing unit 230 may sense a change in state of the reactant on the basis of a time-series record of the reactions made by the reactant to the same predetermined input pattern executed by the same input body.

It is assumed that a characteristic of the reaction of the reactant is affected not only by the input pattern but also by the input body that executes the input pattern.

Therefore, an effect of improving sensing accuracy is expected by performing sensing of a state change for each input body and each predetermined pattern.

Furthermore, a curve L3 and curve L4 in the graph exemplified in FIG. 7 indicate distribution of reaction speed of a non-dementia patient and distribution of reaction speed of a dementia patient, respectively.

Such distribution may be based on pre-stored knowledge (for example, statistical data generated by a research institution).

Furthermore, the state sensing unit 230 according to the present embodiment can sense a sign of a mental illness of the reactant on the basis of the above-described knowledge.

In a case of an example illustrated in FIG. 7, the state sensing unit 230 may sense a sign of dementia of the resident D on the basis of both reaction speed of the resident D to the “greeting” by the nursing care staff G and reaction speed of the resident D to the “greeting” by the nursing care staff H approaching distribution of reaction speed indicated with a dementia patient.

Furthermore, at this time, as exemplified in the lower part of FIG. 7, the presentation control unit 240 may perform control so that a notification regarding a result of the sensing by the state sensing unit 230 is performed.

Moreover, the presentation control unit 240 according to the present embodiment may control presentation of a proposal for improvement for the sensed change in state of the reactant.

In a case of an example illustrated in FIG. 7, the presentation control unit 240 adds a cognitive training program to a schedule of the resident D, and performs control so that presentation of the addition is performed.

According to the presentation control as described above, in addition to early detection of a mental illness or the like, an effect of delaying progression or ameliorating symptoms by proposal for appropriate care is expected.

Note that the presentation control unit 240 may perform control so that the sensed change in state of the reactant is presented to a manager who manages the state of the reactant.

For example, in a case where the reactant is a resident of a nursing home, the above-described manager may be a staff member or the like of the nursing home.

Furthermore, in a case of an example illustrated in FIG. 8, the interface displays a graph illustrating time-series records of two feature values of “voice tone” and “drowsiness” for a certain input pattern, and a notification regarding a sign of depression sensed by the state sensing unit 230 on the basis of the record.

As described above, the state sensing unit 230 according to the present embodiment can also sense a sign of a mental illness on the basis of feature values of two or more reactions.

Furthermore, as illustrated in FIG. 8, the presentation control unit 240 according to the present embodiment may perform control so that a proposal for digital therapeutics (DTx) is presented on the basis of the result of the sensing by the state sensing unit 230.

At this time, for example, the presentation control unit 240 may select an improvement idea such as DTx proposed according to various attributes of the reactant, for example. The presentation control unit 240 may include an age of the reactant in the attributes described above.

For example, the presentation control unit 240 may perform control so that DTx is proposed for reactants in their thirties or younger, recommendation of visiting a hospital and a reward therefor are presented for reactants in their forties to fifties, and a community is introduced for reactants in their sixties or older.

Note that examples of the attributes of a reactant include age, gender, hometown, hobby, personality, and the like.

As described above, the state sensing unit 230 according to the present embodiment can sense a sign of a mental illness or the like on the basis of the time-series records of the feature values of the reactions made by the reactant to the predetermined input pattern.

However, in order to perform sensing as described above, knowledge of a sign of a mental illness is required.

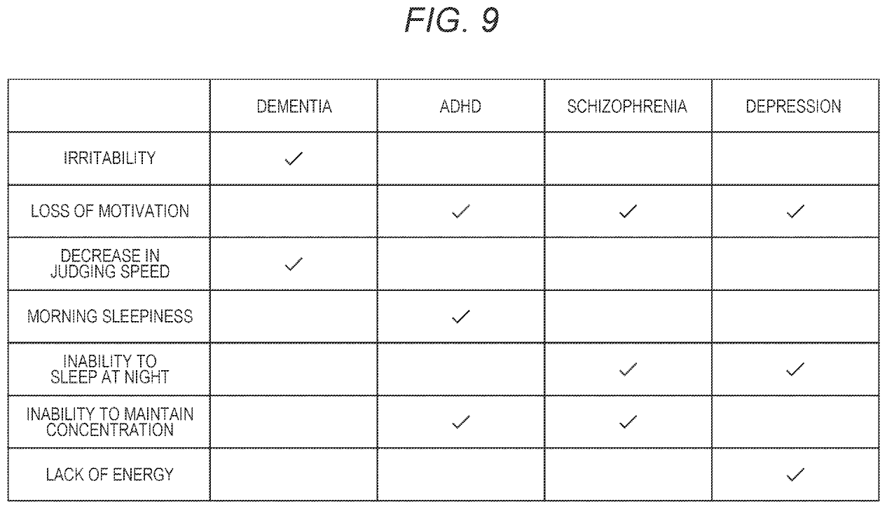

FIG. 9 is a diagram illustrating an example of knowledge of a sign of a mental illness according to the present embodiment.

FIG. 9 illustrates symptoms of the reactant, the symptoms appearing as signs of dementia, attention-deficit hyperactivity disorder, schizophrenia, and depression.

For example, in a case of dementia, a symptom such as irritability or a decrease in judging speed may appear.

Furthermore, for example, in a case of attention-deficit hyperactivity disorder, a symptom such as loss of motivation, morning sleepiness, or inability to maintain concentration may appear.

Furthermore, for example, in a case of schizophrenia, a symptom such as loss of motivation, inability to sleep at night, or inability to maintain concentration may appear.

Furthermore, for example, in a case of depression, a symptom such as loss of motivation, inability to sleep at night, or lack of energy may appear.

Knowledge of a sign of a mental illness as described above may be set in advance on the basis of various statistical data, for example.

Meanwhile, the system according to the present embodiment can also automatically accumulate knowledge of signs of a mental illness as described above.

For this purpose, the system according to the present embodiment may use information of a result of a diagnosis of a mental illness or the like by a physician (hereinafter, simply referred to as diagnostic information).

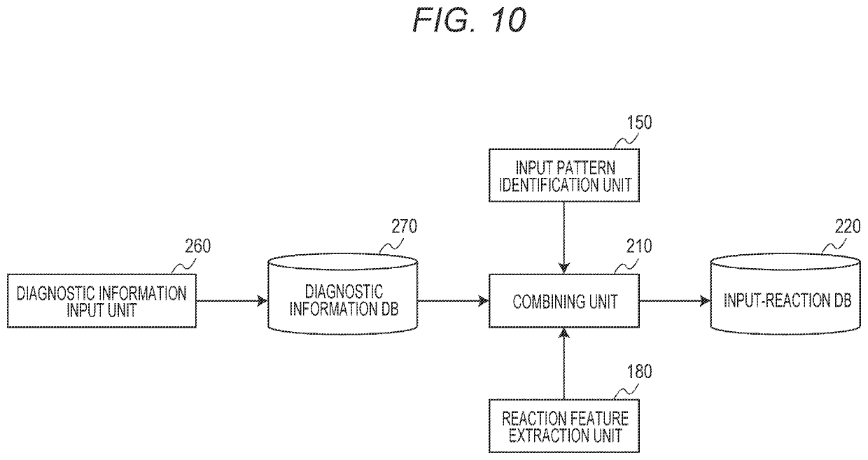

FIG. 10 is a diagram for describing a configuration of recording of information including diagnostic information according to the present embodiment.

As illustrated in FIG. 10, the system according to the present embodiment may further include a diagnostic information input unit 260 and a diagnostic information DB 270 in addition to the configurations illustrated in FIGS. 2 and 5.

### (Diagnostic Information Input Unit 260)

The diagnostic information input unit 260 according to the present embodiment is configured to input diagnostic information.

For this purpose, the diagnostic information input unit 260 according to the present embodiment includes various input devices such as a keyboard and a mouse.

### (Diagnostic Information DB 270)

The diagnostic information DB 270 according to the present embodiment is a database that stores diagnostic information input via the diagnostic information input unit 260.

Diagnostic information stored in the diagnostic information DB 270 may include information regarding a diagnosed reactant, date and time of diagnosis, a diagnosis result (for example, a type and extent of the mental illness, and a comment by a person who has made the diagnosis), the person who has made the diagnosis, and the like.

The combining unit 210 may further combine diagnostic information in addition to the above-described information regarding the input-reaction as illustrated in FIG. 3, and store the combined information in the input-reaction DB 220.

For example, the combining unit 210 can search the diagnostic information DB 270 by using the name, ID, and the like of the reactant and extract diagnostic information regarding the target reactant.

According to the above-described operation, characteristics of the reaction to the input pattern are accumulated in association with diagnosis results by the physician.

The state sensing unit 230 according to the present embodiment can learn a reaction unique to a reactant diagnosed with a predetermined mental illness by using the information accumulated as described above.

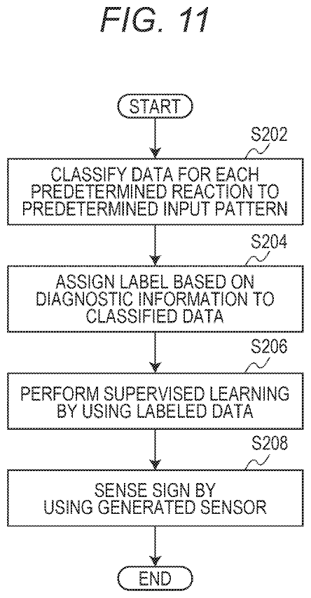

FIG. 11 is a flowchart illustrating an example of a flow of learning by a state sensing unit 230 according to the present embodiment.

In a case of an example illustrated in FIG. 11, first, the state sensing unit 230 classifies the data stored in the input-reaction DB 20 for each predetermined reaction to a predetermined input pattern (S202).

Next, the state sensing unit 230 assigns a label based on diagnostic information (diagnosed or not, name of the diagnosed illness, and the like) to the classified data (S204).

Next, the state sensing unit 230 performs supervised learning by using the labeled data (S206).

Therefore, the state sensing unit 230 can learn a characteristic of a reaction made by a reactant, who is not diagnosed as having a predetermined mental illness, to a predetermined input pattern, and a characteristic of a reaction made by a reactant, who is diagnosed as having the predetermined mental illness, to the predetermined input pattern.

Note that, at this time, it is also possible to cause the state sensing unit 230 to learn characteristics of a change in reaction before and after a diagnosis, by collectively inputting data before and after date and time of diagnosis.

The state sensing unit 230 senses a sign of a mental illness by using a sensor generated by the supervised learning as described above (S208).

Note that, for example, machine learning technology such as a neural network may be used for the supervised learning as described above.

As described above, the state sensing unit 230 according to the present embodiment can sense a change in state of the reactant to be sensed, further on the basis of time-series records of reactions made by another reactant different from the reactant to be sensed.

The above-described another reactant may include an individual diagnosed as having a predetermined state (for example, a mental illness).

Meanwhile, the learning by the state sensing unit 230 according to the present embodiment is not limited to supervised learning.

The state sensing unit 230 according to the present embodiment may perform clustering using data regarding input-reaction including diagnostic information as described above, and may sense a sign of a mental illness on the basis of a result of the clustering.

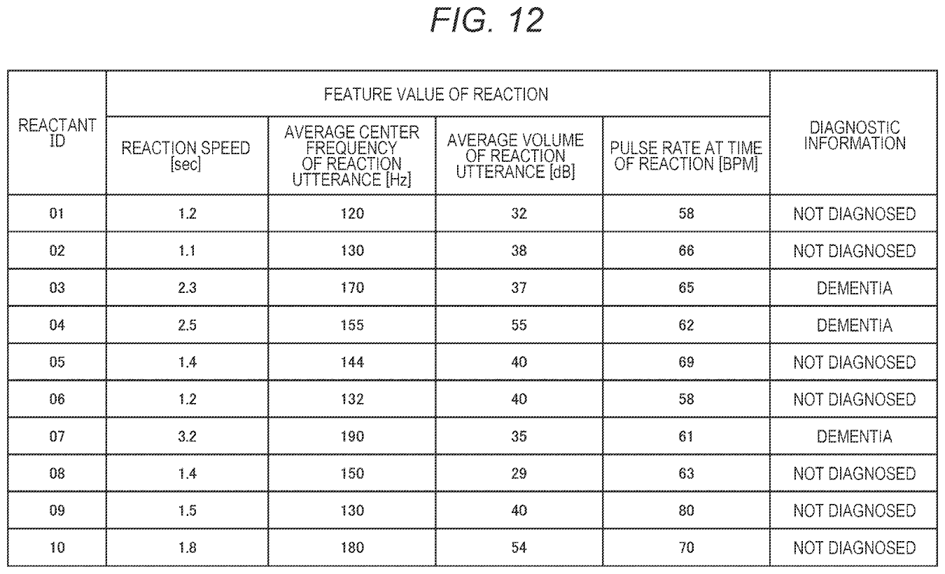

FIG. 12 is a diagram illustrating an example of data used for clustering according to the present embodiment.

In a case of an example illustrated in FIG. 12, each data includes an ID of a reactant reacted to a predetermined input pattern (not illustrated), feature values of various reactions, and diagnostic information (diagnosed or not, name of diagnosed mental illness).

The above-described feature value may include, for example, a reaction speed [sec], an average center frequency of a reaction utterance [Hz], an average volume of the reaction utterance [dB], a pulse rate at a time of the reaction [BPM], and the like.

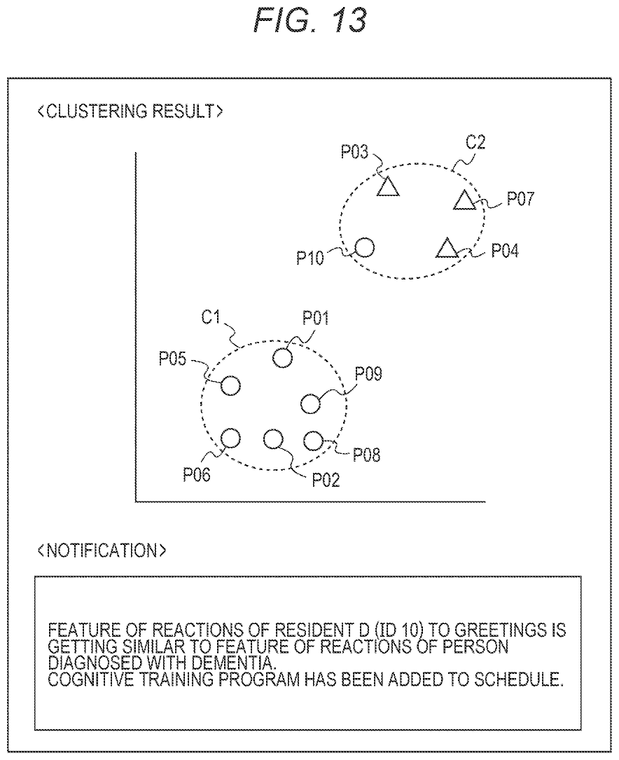

FIG. 13 is a diagram illustrating a result of clustering of data illustrated in FIG. 12 and an example of presentation based on the result.

A graph illustrating a result of the above-described clustering is illustrated in the upper part of FIG. 13. The graph may be obtained by projecting distances between data based on the above-described four feature values in a two-dimensional plane.

Note that plots P01 to P10 in the graph illustrated in FIG. 13 may correspond to reactants ID 01 to ID 10 illustrated in FIG. 12, respectively.

The plots P03, P04, and P07 corresponding to the reactants ID 03, ID 04, and ID 07 diagnosed with dementia, respectively, are indicated by triangles, and the plots corresponding to the another reactant IDs not diagnosed with dementia are indicated by circles.

Furthermore, in the graph illustrated in FIG. 13, two clusters C1 and C2 are formed.

The cluster C1 includes only plots corresponding to the reactant IDs that have not been diagnosed with dementia.

Meanwhile, the cluster C2 includes the plot P10 corresponding to the reactant ID 10 not diagnosed with dementia, in addition to the plots P03, P04, and P07 corresponding to the reactants ID 03, ID 04, and ID 07 diagnosed with dementia, respectively.

In this case, the state sensing unit 230 may determine that the cluster C1 is a set for which a sign of dementia does not appear, on the basis of the cluster C1 including only plots corresponding to reactant IDs that have not been diagnosed with dementia.

Meanwhile, the state sensing unit 230 may determine that the cluster C2 is a set for which a sign of dementia appears, on the basis of most of the plots forming the cluster C2 being plots corresponding to reactant IDs that have been diagnosed with dementia.

Furthermore, the state sensing unit 230 can sense that a sign of dementia appears in the reactant ID 10, on the basis of the cluster C2 including the plot P10 corresponding to the reactant ID 10 that has not actually been diagnosed with dementia by a physician.

On the basis of the state sensing unit 230 sensing that a sign of dementia appears in the reactant ID 10, the presentation control unit 240 may perform control so that presentation of the sensing is performed as illustrated in the lower part of FIG. 13.

The clustering according to the present embodiment has been described above with an example.

Note that the following three types of data may be used for the clustering according to the present embodiment.

Data 1. Data of before and after (for example, from three months before to three months after) a diagnosis regarding a reactant diagnosed as having a predetermined mental illness by an examination by a physician.

Data 2. Data of before and after an examination (for example, from three months before to three months after) regarding a reactant diagnosed as not having a predetermined mental illness by an examination by a physician.

Data 3. All past data of a reactant who has not been examined by a physician (that is, who has not been diagnosed as having a predetermined mental illness).

By utilizing Data 3 that accounts for a large amount of all data, a robust result is easily obtained.

Note that, in a case where Data 1 to Data 3 described above are used, the state sensing unit 230 first performs 2-class unsupervised clustering using a method such as k-means, DBSCAN, or a self-organizing map.

Next, between two clusters, the state sensing unit 230 sets a class including larger amount of Data 1 as a “diagnosed” cluster and sets another as a “not diagnosed” cluster.

Furthermore, for each piece of data corresponding to Data 3, in a case where distance of the feature value of the “diagnosed” cluster is shorter than distance of the “not diagnosed” cluster, it may be set as an alert presentation target (sign sensing).

Meanwhile, the state sensing unit 230 may perform logistic regression (other examples include a neural network, distribution estimation (parametric, non-parametric) of each cluster, or the like) on the two clusters of the “diagnosed” cluster and the “not diagnosed” cluster.

In this case, when Data 3 is substituted into a derived regression equation, values from 0 to 1 can be calculated, and the closer the value is to 1, the closer the characteristic is to the characteristic with diagnosis. By setting a threshold value to 0.9 or the like, it is also possible to perform control so that an alert is to be presented when a more dangerous state occurs.

Next, another function of the presentation control unit 240 according to the present embodiment will be described.

For example, the presentation control unit 240 according to the present embodiment may perform control so that a checklist of signs of a mental illness of the reactant is presented to the user such as the input body.

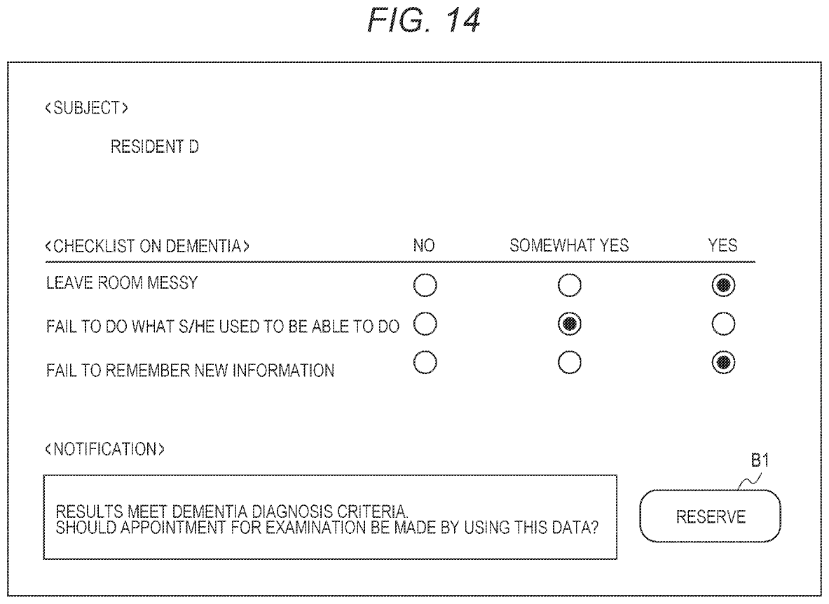

FIG. 14 is a diagram illustrating an example of a checklist according to the present embodiment.

In a case of an example illustrated in FIG. 14, the checklist includes questions asking subjective judgment by the user on signs of dementia with the reactant.

The user, such as an input body to care for the reactant, may input his/her subjective judgment for each question.

The presentation control unit 240 may compare the input information with a predetermined criterion, and in a case where the input information and the predetermined criterion match each other, may, for example, perform control to present a recommendation to book an appointment for an examination by a physician, as illustrated in FIG. 14.

Here, in a case where the user presses a button B1, the presentation control unit 240 may transmit, to the physician, time-series records of reactions of the reactant, results of sensing by the state sensing unit 230, and information of the input checklist, and apply for an appointment.

Note that the checklist as illustrated in FIG. 14 may be presented together with various types of information illustrated in FIGS. 7, 8, 13, and the like.

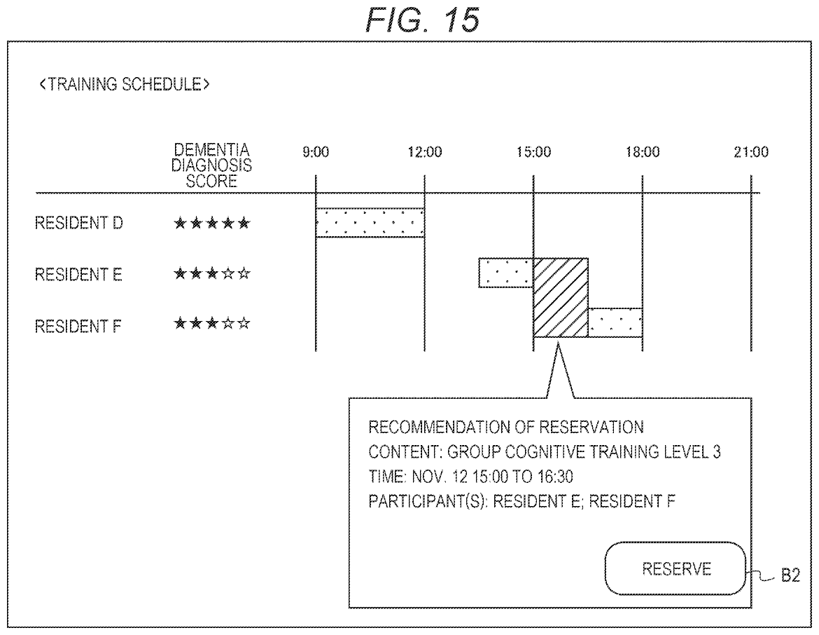

FIG. 15 is an example of an interface for schedule reservation according to the present embodiment.

The presentation control unit 240 according to the present embodiment may control presentation of an interface for managing a schedule of treatment (DTx, training, or the like) in which the reactant participates.

The user such as a nursing care staff may be able to confirm the schedule of the reactant, and newly add, modify, delete, and the like the schedule via an interface as exemplified in FIG. 15.

Furthermore, the presentation control unit 240 according to the present embodiment may control proposal of a new schedule on the basis of free time in the schedule, a diagnostic score (for example, an indication of a progression level of a symptom of a certain mental illness), or the like.

For example, in a case of an example illustrated in FIG. 15, the presentation control unit 240 recommends a new plan for free time common to a resident E and resident F having equivalent diagnostic scores.

According to such a function, it is possible to reduce a burden of schedule management and effectively utilize free time for treatment or the like.

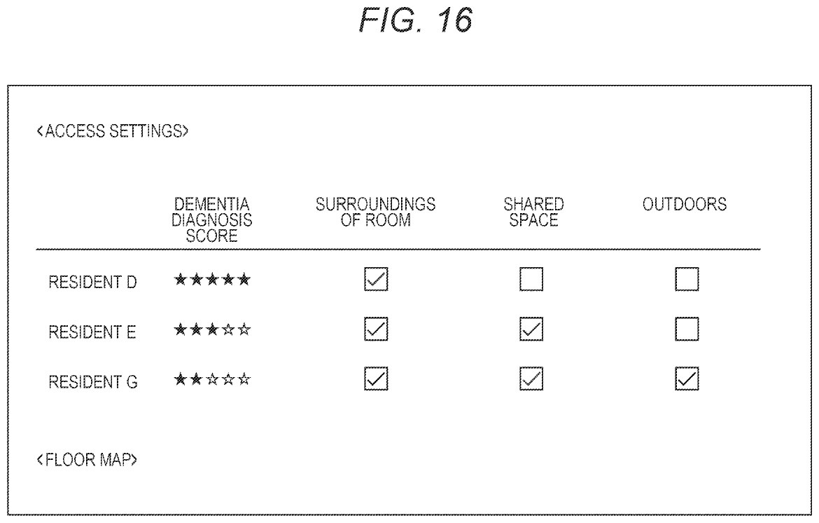

FIG. 16 is an example of an interface for access management of the reactant according to the present embodiment.

The presentation control unit 240 according to the present embodiment may control, for example, presentation of an interface for performing access permission setting for each reactant in the nursing home.

For example, in a case where a symptom of dementia or the like progresses, there is a possibility that the reactant does not return after going out, or that a trouble with another reactant occurs.

Therefore, the administrator may be able to set areas in which access is permitted for each reactant via the user interface controlled by the presentation control unit 240.

For example, in a case of an example illustrated in FIG. 16, an accessible area of the resident D having a level-5 dementia diagnostic score is limited to surroundings of the room.

Meanwhile, an accessible area of the resident E having a level-3 dementia diagnostic score is limited to the surroundings of the room and a shared space.

Meanwhile, a resident G with a level-2 dementia diagnostic score is permitted to access all areas including outdoors.

The above-described settings may be utilized, for example, for automatic locking of a door provided in a facility. In a case where a reactant having no access permission approaches, control such as automatic locking of the door is assumed.

According to the setting and control as described above, safety of the reactants can be maintained.

Note that, in addition to the access settings as described above, the presentation control unit 240 may control presentation of an interface for setting assignment of rooms in a facility.

### 1.4 Application Example

Next, an application example of the system according to the present embodiment will be described.

In the above-described description, there has been described, as a main example, a case where the system according to the present embodiment is applied to sensing of a state change of a resident of a nursing home.

However, the application of the system according to the present embodiment is not limited to the above-described example.

For example, the system according to the present embodiment may be used for assistance of a physician who is not specialized in mental illnesses in a medical institution.

According to the system according to the present embodiment, even a physician who is not familiar with the psychiatric field can notice a change in mental state of a reactant, and therefore, an effect of reducing a possibility of erroneous diagnosis is expected.

For example, presenting to the physician that a reactant who has visited for treatment of heart failure has a tendency of depression allows the physician to first alleviate a symptom of depression and then make a treatment plan such as performing exercise therapy, which is treatment of heart failure.

Furthermore, use of the system according to the present embodiment is not limited to use by health care workers.

The system according to the present embodiment may be used, for example, in a general home, or the like.

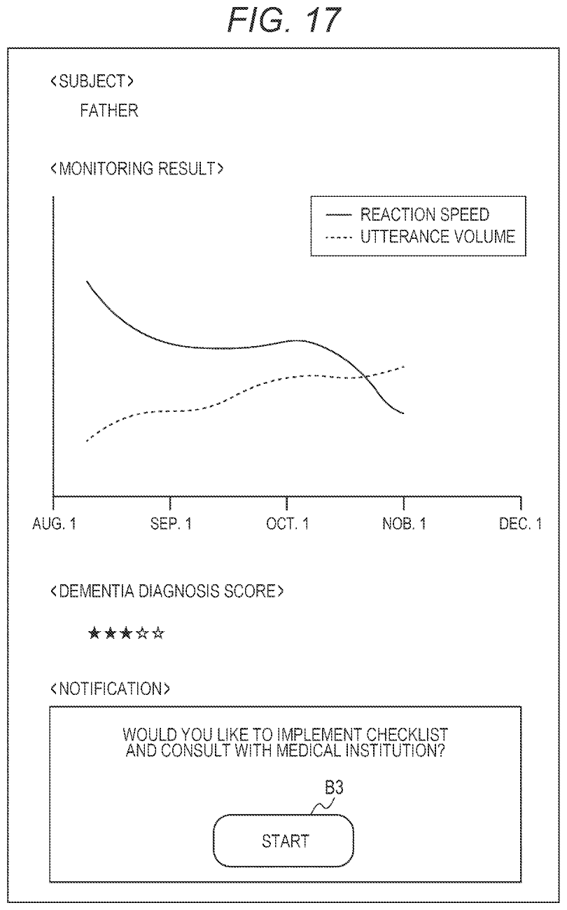

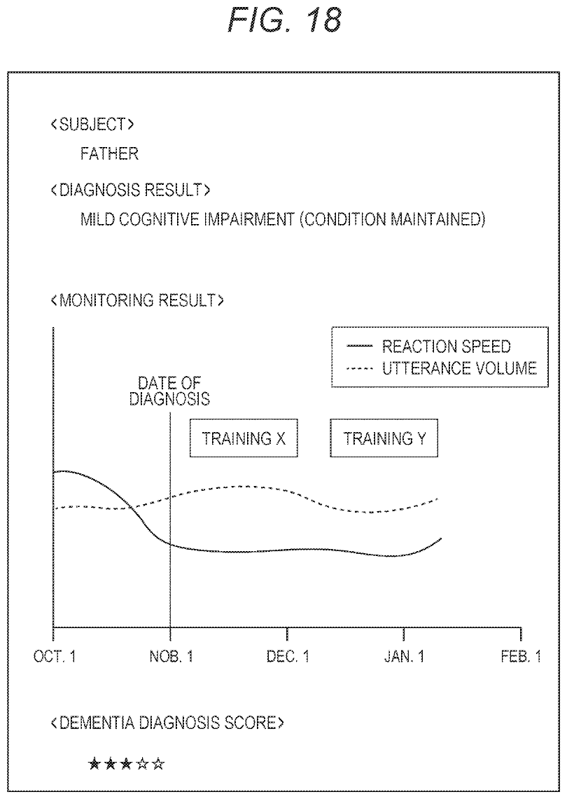

FIGS. 17 and 18 are diagrams illustrating examples of an interface in a case where the system according to the present embodiment is used in a general home.

For example, the interface illustrated in FIG. 17 displays a result of monitoring reactions of a father, a dementia diagnostic score based on the monitoring result, and a notification recommending implementation of a checklist and diagnosis in a medical institution.

For example, a user who is a family member of a subject may press a button B3 to implement the checklist and make a reservation for examination.

Furthermore, an interface illustrated in FIG. 18 displays a monitoring result after having diagnosed in the medical institution with mild cognitive impairment. With this result, a date of diagnosis and a training period performed after diagnosis can be confirmed.

According to the interfaces as illustrated in FIGS. 17 and 18, even a general user who does not have medical knowledge can sense a change in state of a reactant and can continuously monitor the change in state even after diagnosis.

Note that the user who is a family member of the subject may be able to access the interfaces as illustrated in FIGS. 17 and 18 via a family group on an SNS, for example.

Furthermore, the system according to the present embodiment may be applied to an online class, an online meeting, and the like.

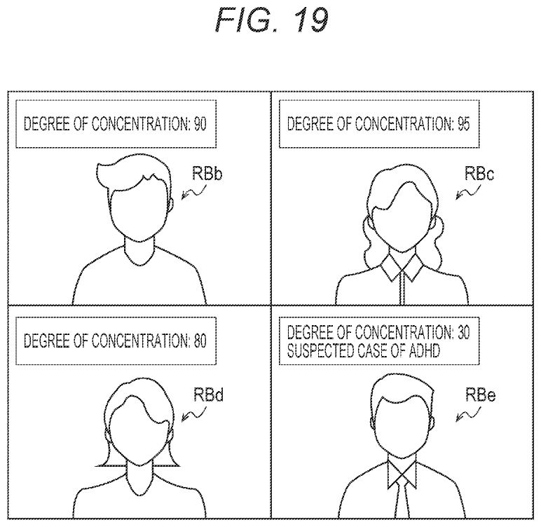

FIG. 19 is a diagram for describing an example in a case where the system according to the present embodiment is applied to an online class or the like.

In a case of an example illustrated in FIG. 19, the presentation control unit 240 performs control to present information regarding a degree of concentration extracted as a reaction to an input pattern (for example, teaching by a lecturer) for each of reactants RBb, RBC, RBd, and RBe who take the online class.

Furthermore, the presentation control unit 240 performs control to present information regarding a sign of ADHD sensed by the state sensing unit 230 on the basis of a change in degree of concentration of the reactant RBe in a predetermined period.

By the lecturer recognizing the above-described information, there will be an increasing chance of providing education suitable for characteristics of a student without lowering motivation of the student.

Note that a symptom of ADHD is not only congenital, but also may be observed by atrophy of a frontal lobe and amygdala. Therefore, under influence of environmental changes (for example, the student has entered an elementary school where students have to stay still, or has entered a university where students have to concentrate on long lectures), a symptom of “looking around frequently with difficulty in concentration” or the like may be easily observed.

Note that the presentation control unit 240 may control presentation of not only an online class but also information regarding a reaction of an employee participating in an online meeting.

The presentation control unit 240 may control, for example, presentation of notification of an employee having a tendency of depression, booking of an appointment of a counseling session, or the like.

Furthermore, the system according to the present embodiment can also be applied to sensing of an abuse.

For example, not only parents but also childcare workers, educators, cram school teachers, and the like may be perpetrators of child abuse, and abuse strongly affects child development, which is a social problem. Moreover, abuse leads to a mental illness such as post-traumatic stress disorder or personality disorder.

Therefore, an effect of protecting current and future safety of a child is expected with sensing of possibility of abuse at an early stage by the system according to the present embodiment.

For example, a person who wishes to deter abuse (if a perpetrator is a parent, the person is a local government, school, or the like, and if a perpetrator is a childcare worker, an educator, or the like, the person is a parent) has a child carry a wearable device to record information regarding inputs and reactions.

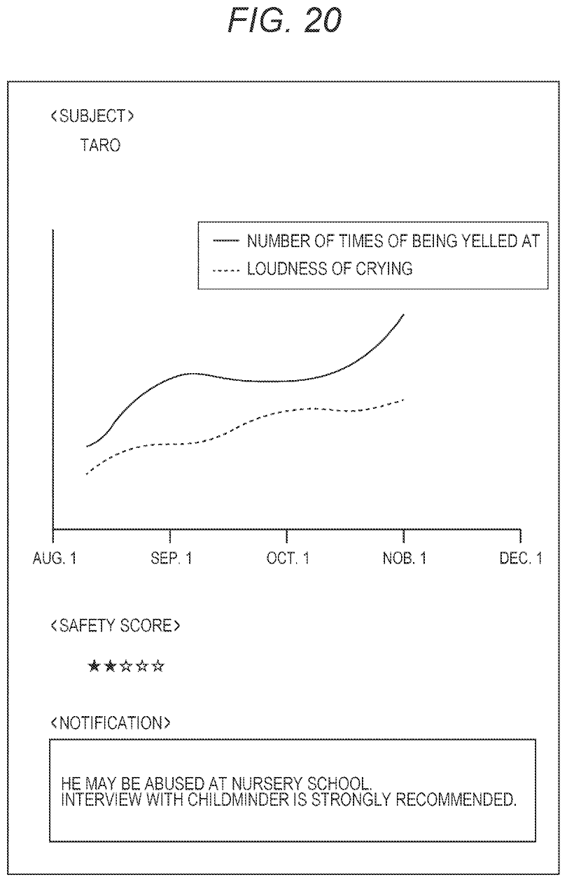

FIG. 20 is a diagram illustrating an example of an interface that presents information regarding sensing of abuse according to the present embodiment.

In a case of an example illustrated in FIG. 20, an interface displays time-series records of the number of times of having been yelled at (input pattern) and loudness of crying as reactions to abuses, a safety score calculated on the basis of the record, and a notification regarding possibility of abuse sensed on the basis of the records.

As an example, yelling can be extracted on the basis of sound quality, utterance content, or the like.

Furthermore, other examples of the reactions include an increase in heart rate, sweating, and utterance content (such as “I'm sorry”) after yelling is detected.

Furthermore, in a case where there is a strong peak in acquired acceleration, it is also possible to consider that the user has been hit (input pattern) in addition to content of acquired sound (sound of beating or utterance of “ouch” or the like).

It is possible to sense an abuse as described above at an early stage and take appropriate measures for protecting safety of a child.

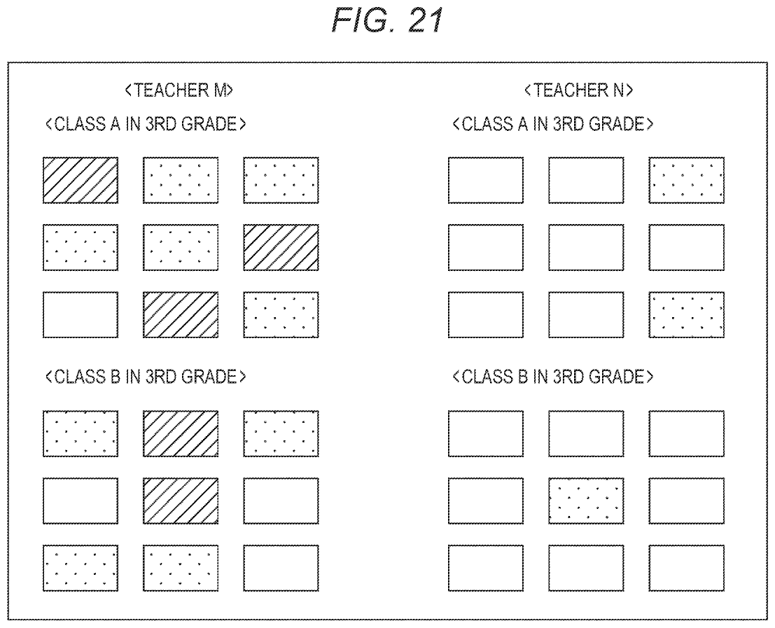

Furthermore, FIG. 21 is an example of an interface in a case of collectively monitoring a plurality of students to know whether or not a specific teacher performs an act that can be regarded as abuse.

In the case of an example illustrated in FIG. 21, the interface displays information indicating strength of negative reactions of students of Class A and Class B in Third grade to words and actions of a teacher M, and strength of negative reactions of students of Class A and Class B in Third grade to words and actions of a teacher N.

Note that, in FIG. 21, each rectangle corresponds to each student, and a pattern of the rectangle represents strength of a negative reaction (diagonal lines: strong, dots: medium, plain: weak).

According to such an interface, it is possible to visualize the strength of characteristics of mental illnesses or the like for each combination of a teacher and a student.

Furthermore, with the above-described visualization, it is possible to sense that a specific teacher has many behaviors related to abuse, that a specific student has received behaviors related to abuse from a plurality of teachers, and the like, and it is possible to cope with the abuse early.

Application of the system 1 according to the present embodiment has been described above with specific examples.

Note that the system 1 according to the present embodiment can be applied to other fields, for example, tracking of a mental state of a person who is subject to compensation for absence from work in health insurance, tracking of cognitive ability of a driver of an automobile, and the like, in addition to the examples described above.

### 2. HARDWARE CONFIGURATION EXAMPLE

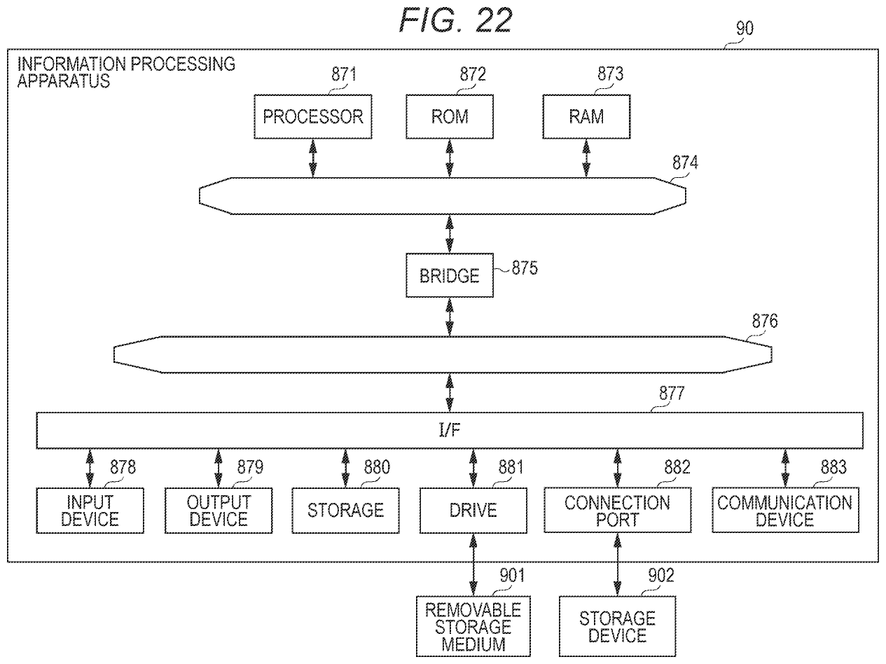

Next, a hardware configuration example of an information processing apparatus 90 according to an embodiment of the present disclosure will be described. FIG. 22 is a block diagram illustrating a hardware configuration example of the information processing apparatus 90 according to an embodiment of the present disclosure. The information processing apparatus 90 may be an apparatus having a hardware configuration equivalent to that of the above-described information processing apparatus 20.

As illustrated in FIG. 22, the information processing apparatus 90 includes, for example, a processor 871, a read only memory (ROM) 872, a random access memory (RAM) 873, a host bus 874, a bridge 875, an external bus 876, an interface 877, an input device 878, an output device 879, a storage 880, a drive 881, a connection port 882, and a communication device 883. Note that the hardware configuration illustrated here is an example, and some of the components may be omitted. Furthermore, components other than the components illustrated here may be further included.

### (Processor 871)

The processor 871 functions as, for example, an arithmetic processing apparatus or a control device, and controls the overall operation of each component or a part thereof on the basis of various programs recorded in the ROM 872, the RAM 873, the storage 880, or a removable storage medium 901.

### (ROM 872, RAM 873)

The ROM 872 is a unit that stores a program read by the processor 871, data used for calculation, or the like. The RAM 873 temporarily or permanently stores, for example, a program read by the processor 871, various parameters that appropriately change when the program is executed, or the like.

### (Host Bus 874, Bridge 875, External Bus 876, and Interface 877)

The processor 871, the ROM 872, and the RAM 873 are mutually connected via, for example, the host bus 874 capable of high-speed data transmission. Meanwhile, the host bus 874 is connected to the external bus 876 having a relatively low data transmission speed via the bridge 875, for example. Furthermore, the external bus 876 is connected with various components via the interface 877.

### (Input Device 878)

As the input device 878, for example, a mouse, a keyboard, a touch panel, a button, a switch, a lever, or the like is used. Moreover, as the input device 878, a remote controller (hereinafter referred to as a remote) capable of transmitting a control signal using infrared rays or other radio waves may be used. Furthermore, the input device 878 includes a sound input device such as a microphone.

### (Output Device 879)

The output device 879 is, for example, a device capable of visually or audibly notifying the user of acquired information, such as a display device such as a cathode ray tube (CRT), an LCD, or an organic EL, an audio output device such as a speaker or a headphone, a printer, a mobile phone, or a facsimile. Furthermore, the output device 879 according to the present disclosure includes various vibration devices capable of outputting tactile stimulation.

### (Storage 880)

The storage 880 is a device for storing various kinds of data. As the storage 880, for example, there is used a magnetic storage device such as a hard disk drive (HDD), a semiconductor storage device, an optical storage device, or a magneto-optical storage device.

### (Drive 881)

The drive 881 is, for example, a device that reads information recorded on the removable storage medium 901 such as a magnetic disk, an optical disk, a magneto-optical disk, or a semiconductor memory, or writes information on the removable storage medium 901.

### (Removable Storage Medium 901)

The removable storage medium 901 is, for example, a DVD medium, a Blu-ray (registered trademark) medium, an HD DVD medium, various semiconductor storage media, or the like. Of course, the removable storage medium 901 may be, for example, an IC card on which a non-contact IC chip is mounted, an electronic device, or the like.

### (Connection Port 882)

The connection port 882 is a port for connecting an external connection device 902 such as a universal serial bus (USB) port, an IEEE 1394 port, a small computer system interface (SCSI), an RS-232C port, or an optical audio terminal.

### (External Connection Device 902)

The external connection device 902 is, for example, a printer, a portable music player, a digital camera, a digital video camera, an IC recorder, or the like.

### (Communication Device 883)

The communication device 883 is a communication device for connecting to a network, for example, a wired or wireless LAN, Bluetooth (registered trademark), or a communication card for Wireless USB (WUSB), a router for optical communication, a router for Asymmetric Digital Subscriber Line (ADSL), or a modem for various communications, or the like.

### 3. CONCLUSION

As described above, the information processing apparatus 20 according to an embodiment of the present disclosure includes the state sensing unit 230 that senses a change in state of the reactant on the basis of a time-series record of the reactions made by the reactant to at least one predetermined input pattern executed by an input body.

Furthermore, one of the characteristics of the above-described predetermined input pattern is an event that repeatedly occurs in an environment in which the reactant lives. According to the above-described configuration, it is possible to sense a sign of a predetermined state at an early stage.

The preferred embodiments of the present disclosure have been described above in detail with reference to the accompanying drawings, but the technical scope of the present disclosure is not limited to such examples. It is obvious that those with ordinary skill in the technical field of the present disclosure may conceive various modifications or corrections within the scope of the technical idea recited in claims, and it is naturally understood that they also fall within the technical scope of the present disclosure.

Furthermore, each step related to the processing described in the present specification is not necessarily processed in time series in the order described in the flowchart or the sequence diagram. For example, each step related to the processing of each device may be processed in an order different from the described order or may be processed in parallel.

Furthermore, the series of processing by each device described in the present specification may be implemented using any of software, hardware, and a combination of software and hardware. The program that constitutes the software is provided inside or outside each device, for example, and is stored in advance in a non-transitory computer readable medium readable by a computer. Then, each program is read into the RAM at the time of execution by the computer, for example, and is executed by various processors. The storage medium is, for example, a magnetic disk, an optical disk, a magneto-optical disk, a flash memory, or the like. Furthermore, the computer program described above may be distributed via, for example, a network without using a storage medium.

Furthermore, the effects described in the present specification are merely exemplary or illustrative, and not restrictive. That is, the technology according to the present disclosure may provide other effects described above that are apparent to those skilled in the art from the description of the present specification, in addition to or instead of the effects described above.

Note that the following configurations also fall within the technical scope of the present disclosure.
(1)
An Information processing apparatus including
a state sensing unit that senses a change in state of a reactant on the basis of a time-series record of reactions made by the reactant to at least one predetermined input pattern executed by an input body,
in which the predetermined input pattern is an event that repeatedly occurs in an environment in which the reactant lives.
(2)
The information processing apparatus according to (1),
in which the predetermined input pattern includes a word or action by the input body to the reactant.
(3)
The information processing apparatus according to (2),
in which the predetermined input pattern includes at least any one of a greeting, request, or question by the input body to the reactant.
(4)
The information processing apparatus according to any one of (1) to (3),
in which the state sensing unit senses a change in mental state of the reactant on the basis of the time-series record of reactions made by the reactant.
(5)
The information processing apparatus according to (4),
in which the state sensing unit senses a sign of a mental illness of the reactant on the basis of the time-series record of reactions made by the reactant.
(6)
The information processing apparatus according to (5),
in which the mental illness includes at least any one of dementia, attention-deficit hyperactivity disorder, schizophrenia, or depression.
(7)
The information processing apparatus according to any one of (1) to (6),
in which the state sensing unit senses a change in state of the reactant on the basis of a time-series record of reactions made by the reactant to a same the predetermined input pattern executed by a same the input body.
(8)
The information processing apparatus according to any one of (1) to (7),
in which the state sensing unit senses a change in state of the reactant to be sensed, further on the basis of a time-series record of reactions made by another reactant different from the reactant to be sensed.
(9)
The information processing apparatus according to (8),
in which the another reactant includes an individual diagnosed as having a predetermined state.
(10)
The information processing apparatus according to any one of (1) to (9),
the information processing apparatus further including a presentation control unit that controls presentation of a result of sensing by the state sensing unit.
(11)
The information processing apparatus according to (10),
in which the presentation control unit performs control so that sensed the change in state of the reactant is presented to a manager who manages the state of the reactant.
(12)
The information processing apparatus according to (10) or (11),
in which the presentation control unit controls presentation of a time-series record of reactions made by the reactant to the predetermined input pattern.
(13)
The information processing apparatus according to any one of (10) to (12),
in which the presentation control unit controls presentation of a proposal for improvement with respect to sensed the change in state of the reactant.
(14)
The information processing apparatus according to any one of (1) to (13),
the information processing apparatus further including an input pattern identification unit that identifies the predetermined input pattern on the basis of sensor information collected for the input body.
(15)
The information processing apparatus according to any one of (1) to (14),
in which the reactant includes at least a care receiver.
(16)
An Information processing method including, by a processor, sensing a change in state of a reactant on the basis of a time-series record of reactions made by the reactant to at least one predetermined input pattern executed by an input body,
in which the predetermined input pattern is an event that repeatedly occurs in an environment in which the reactant lives.
(17)
A program causing a computer to function as an information processing apparatus including a state sensing unit that senses a change in state of a reactant on the basis of a time-series record of reactions made by the reactant to at least one predetermined input pattern executed by an input body,
in which the predetermined input pattern is an event that repeatedly occurs in an environment in which the reactant lives.

### REFERENCE SIGNS LIST

20 Information processing apparatus
110 Input information acquisition unit
120 Input body recognition unit
130 Input feature extraction unit
140 Approach sensing unit
150 Input pattern identification unit
160 Reaction information acquisition unit
170 Reactant recognition unit
180 Reaction feature extraction unit
190 Feature pattern DB
210 Combining unit
220 Input-reaction DB
230 State sensing unit
240 Presentation control unit
250 Presentation unit
260 Diagnostic information input unit
270 Diagnostic information DB

1. An Information processing apparatus comprising
a state sensing unit that senses a change in state of a reactant on a basis of a time-series record of reactions made by the reactant to at least one predetermined input pattern executed by an input body,
wherein the predetermined input pattern is an event that repeatedly occurs in an environment in which the reactant lives.

2. The information processing apparatus according to claim 1,
wherein the predetermined input pattern includes a word or action by the input body to the reactant.

3. The information processing apparatus according to claim 2,
wherein the predetermined input pattern includes at least any one of a greeting, request, or question by the input body to the reactant.

4. The information processing apparatus according to claim 1,
wherein the state sensing unit senses a change in mental state of the reactant on a basis of the time-series record of reactions made by the reactant.

5. The information processing apparatus according to claim 4,
wherein the state sensing unit senses a sign of a mental illness of the reactant on a basis of the time-series record of reactions made by the reactant.

6. The information processing apparatus according to claim 5,
wherein the mental illness includes at least any one of dementia, attention-deficit hyperactivity disorder, schizophrenia, or depression.

7. The information processing apparatus according to claim 1,
wherein the state sensing unit senses a change in state of the reactant on a basis of a time-series record of reactions made by the reactant to a same the predetermined input pattern executed by a same the input body.

8. The information processing apparatus according to claim 1,
wherein the state sensing unit senses a change in state of the reactant to be sensed, further on a basis of a time-series record of reactions made by another reactant different from the reactant to be sensed.

9. The information processing apparatus according to claim 8,
wherein the another reactant includes an individual diagnosed as having a predetermined state.

10. The information processing apparatus according to claim 1,
the information processing apparatus further comprising a presentation control unit that controls presentation of a result of sensing by the state sensing unit.

11. The information processing apparatus according to claim 10,
wherein the presentation control unit performs control so that sensed the change in state of the reactant is presented to a manager who manages the state of the reactant.

12. The information processing apparatus according to claim 10,
wherein the presentation control unit controls presentation of a time-series record of reactions made by the reactant to the predetermined input pattern.

13. The information processing apparatus according to claim 10,
wherein the presentation control unit controls presentation of a proposal for improvement with respect to sensed the change in state of the reactant.

14. The information processing apparatus according to claim 1,
the information processing apparatus further comprising an input pattern identification unit that identifies the predetermined input pattern on a basis of sensor information collected for the input body.

15. The information processing apparatus according to any of claim 1,
wherein the reactant includes at least a care receiver.

16. An Information processing method comprising, by a processor, sensing a change in state of a reactant on a basis of a time-series record of reactions made by the reactant to at least one predetermined input pattern executed by an input body,
wherein the predetermined input pattern is an event that repeatedly occurs in an environment in which the reactant lives.

17. A program causing a computer to function as an information processing apparatus comprising a state sensing unit that senses a change in state of a reactant on a basis of a time-series record of reactions made by the reactant to at least one predetermined input pattern executed by an input body,
wherein the predetermined input pattern is an event that repeatedly occurs in an environment in which the reactant lives.
